(19) United States
(12) Patent Application Publication
(10) Pub. No.: US 2006/0008416 A1
(43) Pub. Date: Jan. 12, 2006

(54) High resolution metabolic brain imaging

(76) Inventor: Scott A. Small, New York, NY (US)

Correspondence Address: COOPER & DUNHAM, LLP, 1185 AVENUE OF THE AMERICAS, NEW YORK, NY, 10036, US

(21) Appl. No.: 10/981,108

(22) Filed: Nov. 3, 2004

Related U.S. Application Data
(60) Provisional application No. 60/517,419, filed on Nov. 4, 2003.

(51) Int. Cl. A61K 49/08

(52) U.S. Cl. 424/9.36

(57) ABSTRACT

This invention provides a method for determining whether a subject is afflicted with Alzheimer's disease by comparing the metabolic activity of the subject's hippocampal entorhinal cortex with that of a second region of the subject's brain. This invention further provides a method for determining whether a subject is afflicted with Alzheimer's disease by comparing the metabolic activity of the subject's hippocampal entorhinal cortex determined at a first and second time point. Finally, this invention provides a method for determining the amount of blood in a volume of cerebral tissue in vivo, wherein the volume of tissue is 1 mm³ or less.

This application claims priority of U.S. Ser. No. 60/157,419, filed Nov. 4, 2003, the contents of which are hereby incorporated by reference into this application.

This invention was made with support under United States Government Grant Nos. AG08702 and AG00949 from the National Institutes of Health. Accordingly, the United States Government has certain rights in the subject invention.

Throughout this application, certain publications are referenced. Full citations for these publications, as well as additional related references, may be found immediately preceding the claims. The disclosures of these publications are hereby incorporated by reference into this application in order to more fully describe the state of the art as of the date of the invention described and claimed herein.

### BACKGROUND OF THE INVENTION

As the development of drugs for Alzheimer's disease continues, there is an urgent need to diagnose Alzheimer's disease at its earliest stages, when pathology is restricted to the hippocampal formation and the disease presents itself as mild memory decline. There is an equally important need to map the progression of Alzheimer's disease pathology over time so that new drugs may be tested. The existence of other causes of age-related memory decline, which also target the hippocampus and mimic early Alzheimer's disease, is the main reason why there is no accurate diagnosis of Alzheimer's disease in its earliest stage.

In principle, this diagnostic ambiguity can be resolved by relying on the microanatomy of the hippocampal formation and on mechanisms of hippocampal dysfunction. The hippocampal formation is a complex structure made up of small and interconnecting subregions, and evidence suggests that early Alzheimer's disease and non-Alzheimer's disease memory decline target different hippocampal subregions.

### SUMMARY OF THE INVENTION

This invention provides a method for determining whether a subject is afflicted with Alzheimer's disease by comparing (a) the metabolic activity of the subject's hippocampal entorhinal cortex with (b) the metabolic activity of a second region of the subject's brain, which second region has metabolic activity that is known not to diminish as a result of Alzheimer's disease, wherein a metabolic activity of the hippocampal entorhinal cortex which is less than or equal to that of the second region of the subject's brain indicates that the subject is afflicted with Alzheimer's disease.

This invention further provides a method for determining whether a subject is afflicted with Alzheimer's disease by comparing the metabolic activity of the subject's hippocampal entorhinal cortex determined at a first time point with that determined at a second time point following the first time point by a suitable period of time, wherein the metabolic activity at the second time point being lower than that at the first time point indicates that the subject is afflicted with Alzheimer's disease.

This invention also provides a method for determining the amount of blood in a volume of cerebral tissue in vivo, wherein the volume of tissue is 1 mm³ or less, comprising: (a) acquiring a first image of the volume of tissue; (b) administering a contrast agent to the volume of tissue; (c) acquiring a second image of the volume of tissue, wherein the second image is acquired at least four minutes after the administration of the contrast agent; and (d) determining the cerebral blood volume of the volume of tissue based on the first and second images.

Finally, this invention provides a method for determining whether, in a subject afflicted with memory loss, the memory loss is due to a cause other than Alzheimer's disease comprising comparing (a) the metabolic activity of the subject's hippocampal entorhinal cortex with (b) the metabolic activity of a second region of the subject's brain, which second region has metabolic activity that is known not to diminish as a result of Alzheimer's disease, wherein a metabolic activity of the second region which is less than that of the entorhinal cortex indicates that the memory loss of the subject is due to a cause other than Alzheimer's disease.

### BRIEF DESCRIPTION OF THE FIGURES

FIG. 1

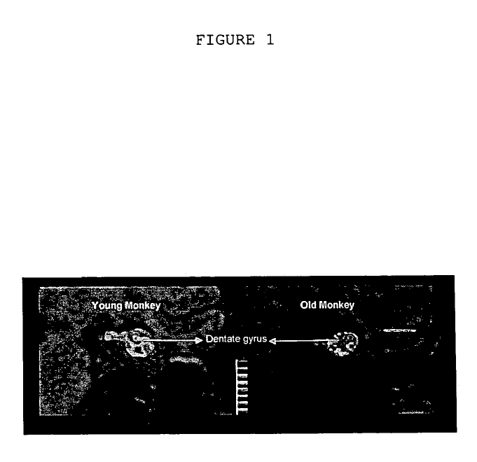

Gadolinium-induced changes in MRI signal, a measure of cerebral blood volume (CBV) and a correlate of oxygen metabolism, is diminished in the dentate gyrus of an old monkey

FIG. 2

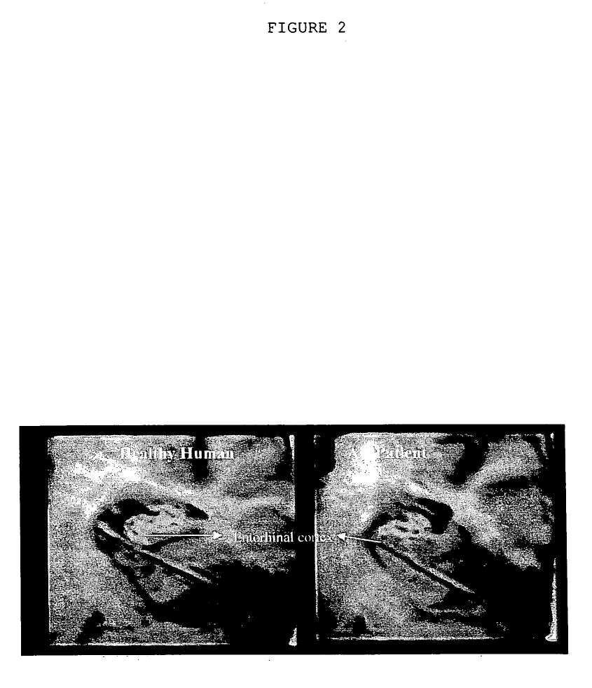

Gadolinium-induced changes in MRI signal, a measure of cerebral blood volume (CBV) and a correlate of oxygen metabolism, is diminished in the entorhinal cortex in a patient with Alzheimer's disease.

### DETAILED DESCRIPTION OF THE INVENTION

Terms

As used in this application, except as otherwise expressly provided herein, each of the following terms shall have the meaning set forth below.

As used herein, “administering” an agent can be effected or performed using any of the various methods and delivery systems known to those skilled in the art. The administering can be performed, for example, intravenously, via cerebrospinal fluid, orally, nasally, via implant, transmucosally, transdermally, intramuscularly, and subcutaneously.

As used herein, “cerebral blood volume” shall mean (i) the volume of blood present in a volume of cerebral tissue, or (ii) a quantitative value (e.g. 1 μm³) correlative either with the volume of blood present in a volume of cerebral tissue and/or with the metabolic activity in that volume of cerebral tissue.

As used herein, “contrast agent” shall mean, where used with respect to brain imaging, any substance administrable to a subject which results in an intravascular enhancement. Examples of contrast agents include paramagnetic substances used in magnetic resonance imaging (such as deoxyhemoglobin or gadolinium).

As used herein, “hippocampal subregion” shall mean any of the nodes of the hippocampus, i.e. entorhinal cortex, CA subfields, caudate nucleus, dentate gyrus and subiculum.

As used herein, “imaging” shall mean the production of a clinically useful image of a subject or portion thereof using, for example, x-rays, ultrasound, computed tomography such as single proton emission computed tomography (SPECT), positron emission tomography (PET), magnetic resonance such as functional magnetic resonance imaging (fMRI), thermography, cross-sectional imaging, or ultra-sonography.

As used herein, “metabolic activity” shall include, without limitation, the chemical changes that occur in a living cell, such as ATP production, which correlate with the cell's energy consumption.

As used herein, “resting metabolic activity” shall include, without limitation, the minimal metabolic activity a cell requires to function properly. In the case of a neuron, for example, resting metabolic activity includes the minimal metabolic activity required to support processes required for normal neuronal function such as signal transduction, second messenger cascades, protein synthesis, axonal transport, synaptic release and synaptogenesis.

As used herein, “subject” s hall mean any animal, such as a primate (e.g. monkey), mouse, rat, guinea pig or rabbit. In the preferred embodiment, the subject is a human.

As used herein, a “suitable period of time” separating first and second time points for determining metabolic activity of a subject's hippocampal entorhinal cortex shall mean any amount of time sufficient to permit a change in the cerebral blood volume in all or a portion of the subject's entorhinal cortex. In the preferred embodiment, the suitable period of time is at least two months.

### EMBODIMENTS OF THE INVENTION

This invention provides a method for determining whether a subject is afflicted with Alzheimer's disease by comparing (a) the metabolic activity of the subject's hippocampal entorhinal cortex with (b) the metabolic activity of a second region of the subject's brain, which second region has metabolic activity that is known not to diminish as a result of Alzheimer's disease, wherein a metabolic activity of the hippocampal entorhinal cortex which is less than or equal to that of the second region of the subject's brain indicates that the subject is afflicted with Alzheimer's disease. In the preferred embodiment, the subject is human.

In one embodiment of the instant method, the metabolic activity is resting metabolic activity. The metabolic activity may be determined by, for example, magnetic resonance imaging (MRI), positron emission tomography (PET) or single proton emission computed tomography (SPECT).

The second region of the subject's brain measured in step (b) of the instant method may be, but is not limited to, the hippocampal dentate gyrus or caudate nucleus.

The metabolic activities of the instant method may be determined by measuring the metabolic activity of tissue having a volume of 1 mm³ or less. The metabolic activity can be represented by, for example, cerebral blood volume.

In further embodiments, the metabolic activity of the subject's hippocampal entorhinal cortex is no more than 90%, 80%, 70%, 60% or 50% of the metabolic activity of the second region of the subject's brain.

This invention further provides a method for determining whether a subject is afflicted with Alzheimer's disease by comparing the metabolic activity of the subject's hippocampal entorhinal cortex determined at a first time point with that determined at a second time point following the first time point by a suitable period of time, wherein the metabolic activity at the second time point being lower than that at the first time point indicates that the subject is afflicted with Alzheimer's disease. In the preferred embodiment, the subject is human.

In one embodiment of the instant method, the metabolic activity is resting metabolic activity. The metabolic activity may be determined by, for example, magnetic resonance imaging (MRI), positron emission tomography (PET) or single proton emission computed tomography (SPECT).

The metabolic activities of the instant method may be determined by measuring the metabolic activity of tissue having a volume of 1 mm³ or less. The metabolic activity may be represented by, for example, cerebral blood volume.

This invention also provides a method for determining the amount of blood in a volume of cerebral tissue in vivo, wherein the volume of tissue is 1 mm³ or less, comprising: (a) acquiring a first image of the volume of tissue; (b) administering a contrast agent to the volume of tissue; (c) acquiring a second image of the volume of tissue, wherein the second image is acquired at least four minutes after the administration of the contrast agent; and (d) determining the cerebral blood volume of the volume of tissue based on the first and second images.

In one embodiment of the instant method, the volume of tissue is within one or more hippocampal subregions.

Finally, this invention provides a method for determining whether, in a subject afflicted with memory loss, the memory loss is due to a cause other than Alzheimer's disease comprising comparing (a) the metabolic activity of the subject's hippocampal entorhinal cortex with (b) the metabolic activity of a second region of the subject's brain, which second region has metabolic activity that is known not to diminish as a result of Alzheimer's disease, wherein a metabolic activity of the second region which is less than that of the entorhinal cortex indicates that the memory loss of the subject is due to a cause other than Alzheimer's disease.

In one embodiment of the instant method, the metabolic activity is resting metabolic activity. The metabolic activity may be determined by, for example, magnetic resonance imaging (MRI), positron emission tomography (PET) or single proton emission computed tomography (SPECT).

The second region of the subject's brain measured in step (b) of the instant method may be, but is not limited to, the hippocampal dentate gyrus or caudate nucleus.

The metabolic activities of the instant method may be determined by measuring the metabolic activity of tissue having a volume of 1 mm³ or less. The metabolic activity can be represented by, for example, cerebral blood volume.

Images may be acquired by, for example, magnetic resonance imaging (MRI), positron emission tomography (PET) or single proton emission computed tomography (SPECT). In the preferred embodiment, images are acquired by functional magnetic resonance imaging (fMRI).

The contrast agent may be endogenous, such as deoxyhemoglobin, or exogenous, such as gadolinium. In the preferred embodiment, the contrast agent is gadolinium.

Contrast agents, their methods of administration, and methods of imaging using same are well known in the art, as described more fully in Kuppusamy, K., et al., Radiology (1996) 201(1): p. 106-112, Losert, C., et al., Magn. Reson. Med. (2002) 48: p. 271-277 and Ogawa, S., et al., Magn Reson Med (1990) 14(1): p. 68-78.

This invention is illustrated in the Experimental Details section which follows. This section is set forth to aid in an understanding of the invention but is not intended to, and should not be construed to limit in any way the invention as set forth in the claims which follow thereafter.

Experimental Details

Introduction

Prior imaging studies in humans have suggested that select subregions of the hippocampal formation are preferentially vulnerable to normal aging. The presence of early Alzheimer's disease can never be excluded in human subjects, however, and so the true locus of normal aging had remained unknown. This issue is resolved here by imaging rhesus monkeys. Like all mammals, monkeys experience age-related decline in hippocampal function, yet monkeys do not develop Alzheimer's disease.

Thus, mapping age-related hippocampal dysfunction in monkeys is free of the diagnostic ambiguities that confound human studies, allowing the hippocampal subregions vulnerable to aging to be isolated.

Synopsis

These experiments show that aging, like other causes of dysfunction in the hippocampal formation, does not affect the hippocampal circuit diffusely. Rather, aging predominately targets the dentate gyrus. Although relatively straightforward, when interlocked with other findings, this information is critical for drawing firm conclusions about the aging brain. First, the positive finding in the dentate gyrus shows that age-related decline in this subregion can occur in the absence of Alzheimer's disease, and provides the needed confirmation absent from prior data. Second, the negative finding in the entorhinal cortex reaffirms other imaging and histological results showing that Alzheimer's disease pathology preferentially targets the entorhinal cortex over the dentate gyrus. These results show that age-related hippocampal dysfunction is not exclusively caused by early Alzheimer's disease, and suggest methods that can differentially diagnose these separate causes.

Weili Lin, et al. developed a model explaining the correlation between signal change in T1-weighted gradient echo images induced by an exogenous contrast agent and regional cerebral blood volume (CBV). The signal difference, before and after gadolinium, measured from pixels within a region-of-interest is divided by the signal differences measured from pixels within the sagittal sinus. Because, by definition, the sagittal sinus has a cerebral blood volume of 100%, using it to normalize signal difference provides quantitative cerebral blood volume measurements. Post-acquisition methods are necessary to assure that signal from large vessels do not confound the measurement of capillary-based cerebral blood volume. Since cerebral blood volume in tissue cannot exceed 10%, any voxel that is found to have a cerebral blood volume value of 10% or larger is excluded from analysis. There are now three MRI-based approaches that generate high-resolution maps of hippocampal dysfunction: resting T2*-weighted maps, oxygen-based CBV maps, and gadolinium-based CBV maps. Detailed below are experiments generating gadolinium-based CBV maps of hippocampal dysfunction in monkeys and humans, and oxygen-based CBV maps of hippocampal dysfunction in transgenic mice.

Materials and Methods

I. Gadolinium-Based CBV maps of Hippocampal Dysfunction in Monkeys

Subjects

Five young rhesus monkeys less than 14 years of age and 4 old monkeys greater than 25 years of age were used. Memory was assessed with a delayed non-match to sample test, a test of hippocampal function. On average, older monkey performed poorer than young monkeys (t=3.3; p=0.03).

Imaging

Three dimensional T1-weighted oblique images perpendicular to the long axis of the hippocampal formation were acquired on a 1.5 tesla magnet (TR=50 ms; TE=5 ms; flip angle=35 degrees; in plane resolution=0.62 mm×0.62 mm; slice thickness 2 mm) before and 4 minutes after IV administration of a standard dose of Omniscan. Post and pre contrast images were subtracted and the difference in signal intensity was measured from the sagittal sinus. The difference images were then divided by the difference in the sagittal sinus and multiplied by 100 to yield percent CBV maps. Pixels with a value greater than 10% were excluded from analysis. Anatomical landmarks were used to identify the entorhinal cortex, the subiculum, the CA subfield, and the dentate gyrus. The average signal from these regions-of-interest was measured on an individual-by-individual basis, tabulated, and used for group data analysis.

II. Gadolinium-Based CBV Maps of Hippocampal Dysfunction in Humans

Subjects

Three young healthy subjects (mean age=32), and one 84 year-old patient with clinically diagnosed probable Alzheimer's disease with moderate dementia were used.

Imaging

Three-dimensional T1-weighted images were acquired on a 1.5 tesla magnet (TR=20 ms; TE=6 ms; flip angle=25 degrees; in plane resolution=0.86 mm×0.86 mm; slice thickness=4 mm) before and 4 minutes after IV administration of a standard dose of Omniscan. The images were processed as in the monkeys.

III. Oxygen-Based CBV Maps of Hippocampal Dysfunction in Mice

Subjects

Mice whose neuronal expression of the 695-amino acid isoform of hAPP (hAPP695) was directed by the prion protein (PrP) promoter fused to an HAPP cDNA (line TgCRND8) were used. In order to insure a functional deficit, these mice were imaged at around 6 months of age, and again when the mice began laying down amyloid plaques and experiencing memory dysfunction.

Imaging

In preparation for imaging, all mice were anesthetized. A 9.4 tesla Bruker magnet was used to acquire T2*-weighted images (TR/TE=300/8, flip angle=30, NEX=8, inplane resolution=0.1 mm, slice thickness=0.7 mm) on and off 100% oxygen. Pulse oximetry was used to monitor oxygen levels, and at baseline the oxygenation level of all mice was 85-95%. Percent change in signal intensity on and off oxygen was calculated, and anatomical landmarks were used to identify regions of interest overlying each hippocampal subregion: the entorhinal cortex, the dentate gyrus, the CA3 and CA1 subfields, and the subiculum. Percent change in signal for each subregion, a measure of relative CBV, was normalized against percent change in the basal vein and used in a multivariate analysis of variance where genotype was included as the independent factor and age and group were included as covariates.

Results

I. Gadolinium-Based CBV Maps of Hippocampal Dysfunction in Monkeys

Imaging results revealed that compared to the young monkeys the older monkeys had significantly diminished CBV in the dentate gyrus (F=9.5; p=0.018) and a trend toward significance was observed in the subiculum. Furthermore, analysis revealed a significant negative correlation between age and CBV in the dentate gyrus (beta=−8.0; p=0.009). No between-group difference was observed in the entorhinal cortex and the CA subfields. Importantly, for diagnostic purposes, logistic regression analysis revealed that CBV from the dentate gyrus distinguished old and young monkeys (p 0.008), with an overall accuracy of 90%.

II. Gadolinium-Based CBV Maps of Hippocampal Dysfunction in Humans

Despite the severe atrophy observed in the Alzheimer's disease patient, there was sufficient tissue to generate ROIs around the entorhinal cortex and other hippocampal subregions. Compared to all three young healthy subjects, the Alzheimer's disease patient had lower signal in all hippocampal subregions. More importantly, the pattern of signal within the patient's hippocampus showed that his entorhinal cortex has the lowest CBV.

III. Oxygen-Based CBV Maps of Hippocampal Dysfunction in Mice

Imaging results revealed that compared to non-transgenic controls, transgenic mice have significantly diminished CBV in the entorhinal cortex (F=9.6; p=0.012) and the CA3 (F=9.4, p=0.018) subregions. No difference was observed in the subiculum or the dentate gyrus.

Discussion

All mammals develop age-related memory decline, but only humans develop age-related memory decline caused by early Alzheimer's disease. Thus, non-humans can only develop non-Alzheimer's disease age-related memory decline.

In the current study, maps of hippocampal dysfunction are generated and show that rhesus monkeys with age-related memory decline have dysfunction restricted to the dentate gyrus and maybe the subiculum. Importantly, MRI measures of the entorhinal cortex did not decline in aged monkeys. By definition, this pattern of hippocampal dysfunction reflects non-Alzheimer's disease memory decline and recapitulates the pattern of hippocampal decline we observed in humans. Beyond validating that non-Alzheimer's disease memory decline targets select hippocampal subregions, the data analysis shows that CBV measures have sufficient power to diagnose dysfunction on an individual basis. However, showing that an Alzheimer's disease patient has lower hippocampal CBV compared to controls is known. This observation has already been made by other studies.

What is new here is to be able to look at individual hippocampal subregions. Compared to other hippocampal subregions, the Alzheimer's disease patient had lowest CBV in his entorhinal cortex. Thus, this hippocampal pattern is consistent with previous findings. These results show that even a patient with more severe dementia can be imaged with the instant protocol; that motion is not a problem; and that even with severe atrophy the hippocampal subregions may be assessed.

Moreover, the pattern of hippocampal dysfunction in the mice expressing Alzheimer's disease genes is consistent with the pattern observed in humans with suspected early Alzheimer's disease. Thus, taken together, these studies indicate that entorhinal dysfunction is indeed a reliable marker for early Alzheimer's disease.

### REFERENCES

1. Abel, T., et al., Genetic demonstration of a role for PKA in the late phase of LTP and in hippocampus-based long-term memory, Cell (1997) 88(5): p. 615-626.
2. Alberts, B., et al., Molecular Biology of the Cell, (1995) Garland Pub. p. 144.
3. Amaral, D. G. and Witter, M. P., The three-dimensional organization of the hippocampal formation: a review of anatomical data, Neuroscience (1989) 31(3): p. 571-591.
4. Amaral, D. G. and Insausti, R., The Hippocampal Formation, in The Human Nervous System., R. Paxinos, Editor (1990) Academic Press: San Diego.
5. Amaral, D. G., Emerging principles of intrinsic hippocampal organization, Curr. Opin. Neurobiol. (1993) 3(2): p. 225-229.
6. Bandettini, P. A., et al., Characterization of cerebral blood oxygenation and flow changes during prolonged brain activation, Hum. Brain Mapp. (1997) 5(2): p. 93-109.
7. Belliveau, J. W., et al., Functional cerebral imaging by susceptibility-contrast NMR, Magn. Reson. Med. (1990) 14(3): p. 538-546.
8. Benton, A. L., FAS test in Neurosensory Center Comprehensive Examination for Aphasia, (1967) Victoria: B. C. University of Victoria.
9. Blessed, G., Tomlinson, B. E. and Roth, M., The association between quantitative measures of dementia and of senile change in the cerebral grey matter of elderly subjects, Br. J. Psychiatry (1968) 114(512): p. 797-811.
10. Bozzao, A., et al., Diffusion and Perfusion MR imagining in cases of Alzheimer's disease: correlations with cortical atrophy and lesion load, AJNR Am. J. Neuroradiol. (2001) 22(6): p. 1030-1036.
11. Braak, H. and Braak, E., Evolution of the neuropathology of Alzheimer's disease, Acta. Neurol. Scand. Suppl. (1996) 165: p. 3-12.
12. Buschke, H. and Fuld, P. A., Evaluating storage, retention, and retrieval in disordered memory and learning, Neurology (1974) 24(11): p. 1019-1025.
13. Cavazzuti, M., et al., Ketamine effects on local cerebral blood flow and metabolism in the rat, J. Cereb. Blood Flow Metab. (1987) 7(6): p. 806-844.
14. Chen, Y. C., et al., Improved mapping of pharmacologically induced neuronal activation using the IRON technique with superparamagnetic blood pool agents, J. Magn. Reson. Imaging, (2001) 14(5): p. 517-524.
15. Chishti, M. A., et al., Early-onset amyloid deposition and cognitive deficits in transgenic mice expressing a double mutant form of amyloid precursor protein 695, J. Biol. Chem., (2001) 276(24): p. 21562-21570.
16. Cohen, E., Ugurbil, K. and Kim, S., Effect of basal conditions on the magnitude and dynamics and the hemodynamic response, Proceeding of the International Society of Magnetic Resonance in Medicine (2002).
17. Costa, D. C., Pilowsky, L. S. and Ell, P. J., Nuclear medicine in neurology and psychiatry, Lancet, (1999) 354(9184): p. 1107-1111.
18. David, T. L., et al., Calibrated functional MRI: mapping the dynamics of oxidative metabolism, Proc. Natl. Acad. Sci. USA (1998) 95(4): p. 1834-1839.
19. Duff, K., Transgenic mouse models of Alzheimer's disease: phenotype and mechanisms of pathogenis, Biochem. Soc. Symp. (2001) 67: p. 195-202.
20. Duvernoy, H. M., The Human Hippocampus: an atlas of applied anatomy, 2^{nd} Edition (1998) Munich: J. F. Bergman.
21. Eberling, J. L., et al., Cerebral glucose metabolism and memory in aged rhesus macaques, Neurobiol. Aging, (1997) 18(4): p. 437-443.
22. Erecinska, M. and Silver, I. A., ATP and brain function, J. Cereb. Blood Flow Metab., (1989) 9(1): p. 2-19.
23. Freeman, F. M., Rose, S. P. and Scholey, A. B., Two time windows of anisomycin-induced amnesia for passive avoidance training in the day-old chick, Neurobiol. Learn. Mem., (1995) 63(3): p. 291-295.
24. Gallagher, M. and Rapp, P. R., The use of animal models to study the effects of aging on cognition, Annu. Rev. Psychol., (1997) 48: p. 339-70.
25. Gallagher, M., Burwell, R. and Burchinal, M., Severity of spatial learning impairment in aging: development of a leaning index for performance in the Morris water maze, Behav. Neurosci., (1993) 107(4): p. 618-626.
26. Gomez-Isla, T., et al., Profound loss of layer II entorhinal cortex neurons occurs in very mild Alzheimer's disease, J. Neurosci., (1996) 16(14): p. 4491-4500.
27. Gonzalez, R. G., et al., Functional MR in the evaluation of dementia: correlation of abnormal dynamic cerebral blood volume measurements with changes in cerebral metabolism on positron emission tomography with fludeoxyglucose F 18, AJNR Am. J. Neuroradiol., (1995) 16(9): p. 1763-1770.
28. Grober, E. and Kawas, C., Learning and retention in preclinical and early Alzheimer's disease, Psychol. Aging, (1997) 12(1): p. 183-188.
29. Harris, G. J., et al., Dynamic susceptibility contrast MR imaging of regional cerebral blood volume in Alzheimer disease: a promising alternative to nuclear medicine. AJNR Am. J. Neuroradiol., (1998) 19(9): p. 1727-1732.
30. Hoge, R. D., et al., Linear coupling between cerebral blood flow and oxygen consumption in activated human cortex, Proc. Natl. Acad. Sci. USA, (1999) 96(16): p. 9403-9408.
31. Hyder, F., et al., Quantitative functional imaging of the brain: towards mapping neuronal activity by BOLD fMRI, NMR Biomed., (2001) 14(7-8): p. 413-431.
32. Hyder, F., et al., Quantitative multi-modal functional MRI with blood oxygenation level dependent exponential decays adjusted for flow attenuated inversion recovery (BOLDED AFFAIR), Magn. Reson. Imaging, (2000) 18(3): p. 227-235.
33. Jacobs, D. M., et al., Neuropsychological detection and characterization of preclinical Alzheimer's disease [comment], Neurology, (1995) 45(5): p. 957-962.
34. Janus, C., et al., A beta peptide immunization reduces behavioural impairment and plaques in a model of Alzheimer's disease, Nature (2000) 408(6815): p. 979-982.
35. Jarrard, L. E., On the role of the hippocampus in learning and memory in the rat, Behav. Neural. Biol., (1993) 60(1): p. 9-26.
36. Kim, J. J. and Fanselow, M. S., Modality-specific retrograde amnesia of fear, Science (1992) 256(5057): p. 675-677.
37. Kuppusamy, K., et al, In vivo regional cerebral blood volume: quantitative assessment with 3D T1-weighted pre- and postcontrast MR imaging, Radiology (1996) 201(1): p. 106-112.
38. Lin, W. A. Celik and Paczynski, R. P., Regional cerebral blood volume: a comparison of the dynamic imaging and the steady state methods, J. Magn. Reson. Imaging (1999). 9(1): p. 44-52.
39. Lin, W., et al., Quantitative measurements of regional cerebral blood volume using MRI in rats: effects of arterial carbon dioxide tension and mannitol, Magn. Reson. Med. (1997) 38(3): p. 420-428.
40. Lorente de No, R., Studies on the structure of the cerebral cortex II. Continuation of the study of the ammonic system, Journal of Psychology and Neurology (1934) 46: p. 113-117.
41. Losert, C., et al., Oxygen-enhanced MRI of the brain, Magn. Reson. Med. (2002) 48(2): p. 271-277.
42. Losert, C., et al., Oxygen-enhanced MRI of the brain. Magn. Reson. Med. (2002) 48: p. 271-277.
43. Lynch, M. A., et al., Increase in synaptic viscle proteins accompanies long-term potentiation in the dentate gyrus. Neuroscience (1994) 60(1): p. 1-5.
44. Masliah, E., et al., Quantitative synaptic alterations in the human neocortex during normal aging, Neurology (1993) 43(1): p. 67-72.
45. Masliah, E., et al., Synaptic and neuritic alterations during the progression of Alzheimer's disease, Neurosci. Lett. (1994) 174(1): p. 67-72.
46. Masur, D. M., et al., Neuropsychological prediction of dementia and the absence of dementia in healthy elderly persons [see comments], Neurology (1994) 44(8): p. 1427-1432.
47. Mayeux, R., et al, Memory performance in healthy elderly without Alzheimer's disease: effects of time and apolipoprotein-E, Neurobiol. Aging (2001) 22(4): p. 683-689.
48. McKhann, G., et al., Clinical diagnosis of Alzheimer's disease: report of the NINCDS-ADRDA Work Group under the auspices of Department of Health and Human Services Task Force on Alzheimer's Disease, Neurology (1984) 34(7): p. 939-944.
49. Morris, R., Developments of a water-maze procedure for studying spatial learning in the rat, J. Neurosci. Methods (1984) 11(1): p. 47-60.
50. Mucke, L., et al., High-level neuronal expression of abeta 1-42 in wild-type human amyloid protein precursor transgenic mice: synaptotoxicity without plaque formation, J. Neurosci. (2000) 20(11): p. 4050-4058.
51. Ogawa, S., et al., Oxygenation-sensitive contrast in magnetic resonance image of rodent brain at high magnetic fields, Magn. Reson. Med. (1990) 14(1): p. 68-78.
52. Paxinos, G. and Franklin, K., The mouse brain in stereotaxic coordinates (2001) Academic Press.
53. Rapp, P. R. and Gallagher, M., Preserved neuron number in the hippocampus of aged rats with spatial learning deficits, Proc. Natl. Acad. Sci. USA, (1996) 93(18): p. 9926-9930.
54. Rosen, The Rosen Drawing Test (1981) Bronx, N.Y.
55. Siesjo, B., Brain energy metabolism, (1978) New York: Wiley.
56. Small G. W., et al., Cerebral metabolic and cognitive decline in persons at genetic risk for Alzheimer's disease, Proc. Natl. Acad. Sci. USA (2000) 97(11): p. 6037-6042.
57. Small, S., et al., Imaging physiologic dysfunction of individual hippocampal subregions in humans and genetically modified mice, Neuron (2000) 28: p. 653-664.
58. Small, S. A., Age-related memory decline; current concepts and future directions, Archives of Neurology (2001) 58: p. 360-364.
59. Small, S. A., et al., Differential regional dysfunction of the hippocampal formation among elderly with memory decline and Alzheimer's disease, Ann. Neurol. (1999) 45(4): p. 466-472.
60. Small, S. A., et al., Evaluating the function of hippocampal subregions with high-resolution MRI in Alzheimer's disease and aging, Micros. Res. Tech. (2000) 51(1): p. 101-108.
61. Small, S. A., et al., Imagining hippocampal function across the human life span: is memory decline normal or not?, Ann. Neurol. (2002) 51(3): p. 290-295.
62. Sokoloff, L., Cerebral Metabolism and Visualization of Cerebral Activity, in Comprehensive Human Physiology, R. Gregor and U. Windhorst, Editors (1996) Springer-Verlag: New York, p. 579-602.
63. Sperling, R. Functional MRI studies in early Alzheimers disease, International Conference on Alzheimer's disease and Related Disorders (2002) Stockholm, Sweden.
64. Stern, Y., et al., Diagnosis of dementia in a heterogeneous population. Development of a neuropsychological paradigm-based diagnosis of dementia and quantified correction for the effects of education. Arch. Neurol. (1992) 49(5): p. 453-460.
65. Thulborn, K. R., et al., Oxygen dependence of the transverse relaxation time of water protons in whole blood at high field, Biochimica et Biophysica Acta, (1982) 714: p. 265-270.
66. Van Zijil, P. C., et al., Quantitative assessment of blood flow, blood volume and blood oxygenation effects in functional magnetic resonance imaging, Nat. Med. (1998) 4(2): p. 159-167.
67. Wansapura, J. P., et al., NMR relaxation times in the human brain at 3.0 tesla, J. Magna. Reson. Imaging. (1999) 9(4): p. 531-538.
68. Wechsler, D., WAIS-R manual, The Psychological Corporation (1981) New York.
69. Zhao, X., et al., Transcriptional profiling reveals strict boundaries between hippocampal subregions, J. Comp. Neurol. (2001) 441(3): p. 187-196.
70. Zola-Morgan, S. M. and Squire, L. R., The primate hippocampal formation: evidence for a time-limited role in memory storage, Science (1990) 250:(4978) p. 288-290.

What is claimed is:

1. A method for determining whether a subject is afflicted with Alzheimer's disease comprising comparing (a) the metabolic activity of the subject's hippocampal entorhinal cortex with (b) the metabolic activity of a second region of the subject's brain, which second region has metabolic activity that is known not to diminish as a result of Alzheimer's disease, wherein a metabolic activity of the hippocampal entorhinal cortex which is less than or equal to that of the second region of the subject's brain indicates that the subject is afflicted with Alzheimer's disease.

2. The method of claim 1, wherein the subject is human.

3. The method of claim 1, wherein the metabolic activity is resting metabolic activity.

4. The method of claim 1, wherein the metabolic activity is determined through magnetic resonance imaging.

5. The method of claim 1, wherein each of the metabolic activities of steps (a) and (b) is determined by measuring the metabolic activity of tissue having a volume of 1 mm or less.

6. The method of claim 1, wherein the metabolic activity is represented by cerebral blood volume.

7. The method of claim 1, wherein the second region is the hippocampal dentate gyrus.

8. The method of claim 1, wherein the second region is the hippocampal caudate nucleus.

9. The method of claim 1, wherein the metabolic activity of the subject's hippocampal entorhinal cortex is no more than 90% of the metabolic activity of the second region of the subject's brain.

10. The method of claim 1, wherein the metabolic activity of the subject's hippocampal entorhinal cortex is no more than 80% of the metabolic activity of the second region of the subject's brain.

11. The method of claim 1, wherein the metabolic activity of the subject's hippocampal entorhinal cortex is no more than 70% of the metabolic activity of the second region of the subject's brain.

12. The method of claim 1, wherein the metabolic activity of the subject's hippocampal entorhinal cortex is no more than 60% of the metabolic activity of the second region of the subject's brain.

13. The method of claim 1, wherein the metabolic activity of the subject's hippocampal entorhinal cortex is no more than 50% of the metabolic activity of the second region of the subject's brain.

14. A method for determining whether a subject is afflicted with Alzheimer's disease, comprising comparing the metabolic activity of the subject's hippocampal entorhinal cortex determined at a first time point with that determined at a second time point following the first time point by a suitable period of time, wherein the metabolic activity at the second time point being lower than that at the first time point indicates that the subject is afflicted with Alzheimer's disease.

15. The method of claim 14, wherein the subject is human.

16. The method of claim 14, wherein the metabolic activity is resting metabolic activity.

17. The method of claim 14, wherein the metabolic activity is determined through magnetic resonance imaging.

18. The method of claim 14, wherein each of the metabolic activities is determined by measuring the metabolic activity of tissue having a volume of 1 mm³ or less.

19. The method of claim 14, wherein the metabolic activity is represented by cerebral blood volume.

20. A method for determining the amount of blood in a volume of cerebral tissue in vivo, wherein the volume of tissue is 1 mm³ or less, comprising:
(a) acquiring a first image of the volume of tissue;
(b) administering a contrast agent to the volume of tissue;
(c) acquiring a second image of the volume of tissue, wherein the second image is acquired at least four minutes after the administration of the contrast agent; and
(d) determining the cerebral blood volume of the volume of tissue based on the first and second images.

21. The method of claim 20, wherein the volume of tissue is within one or more hippocampal subregions.

22. The method of claim 20, wherein the first and second images are acquired by magnetic resonance imaging.

23. The method of claim 20, wherein the contrast agent is gadolinium.

24. A method for determining whether, in a subject afflicted with memory loss, the memory loss is due to a cause other than Alzheimer's disease comprising comparing (a) the metabolic activity of the subject's hippocampal entorhinal cortex with (b) the metabolic activity of a second region of the subject's brain, which second region has metabolic activity that is known not to diminish as a result of Alzheimer's disease, wherein a metabolic activity of the second region which is less than that of the entorhinal cortex indicates that the memory loss of the subject is due to a cause other than Alzheimer's disease.

25. The method of claim 24, wherein the subject is human.

26. The method of claim 24, wherein the metabolic activity is resting metabolic activity.

27. The method of claim 24, wherein the metabolic activity is determined through magnetic resonance imaging.

28. The method of claim 24, wherein each of the metabolic activities of steps (a) and (b) is determined by measuring the metabolic activity of tissue having a volume of 1 mm³ or less.

29. The method of claim 24, wherein the metabolic activity is represented by cerebral blood volume.

30. The method of claim 24, wherein the second region is the hippocampal dentate gyrus.

31. The method of claim 24, wherein the second region is the hippocampal caudate nucleus.
